(19) United States
(12) Patent Application Publication
(10) Pub. No.: US 2020/0405915 A1
(43) Pub. Date: Dec. 31, 2020

(54) HYDROGEL FOR STIMULATING NEUROTIZATION, OSTEOGENESIS AND ANGIOGENESIS

(71) Applicants: UNIVERSITE DE BORDEAUX, BORDEAUX (FR); INSTITUT POLYTECHNIQUE DE BORDEAUX, TALENCE (FR); INSTITUT NATIONAL DE LA SANTÉ ET DE LA RECHERCHE MÉDICALE, PARIS (FR); CENTRE NATIONAL DE LA RECHERCHE SCIENTIFIQUE, PARIS (FR)

(72) Inventors: JOËLLE AMEDEE, PESSAC (FR); SÉBASTIEN LECOMMANDOUX, CANEJAN (FR); HUGO OLIVEIRA, MACAU (FR); BRUNO PAIVA DOS SANTOS, BORDEAUX (FR); BERTRAND GARBAY, GRADIGNAN (FR); ELISABETH GARANGER, TALENCE (FR)

(21) Appl. No.: 16/976,482

(22) PCT Filed: Feb. 28, 2019
(86) PCT No.: PCT/EP2019/055075

§ 371 Date: Aug. 28, 2020

(30) Foreign Application Priority Data
Feb. 28, 2018 (FR) 1851770

(51) Int. Cl. A61L 27/52; A61L 27/54; A61K 47/42; A61P 19/08; A61P 25/00; C08J 3/24

(52) U.S. Cl. CPC A61L 27/52; A61L 27/54; A61K 47/42; A61P 19/08; A61P 25/00; A61K 9/0024; A61L 2300/25; A61L 2400/06; A61L 2430/02; A61L 2300/414; C08J 3/245

(57) ABSTRACT

The present invention relates to a hydrogel useful to promote neurotization, osteogenesis and angiogenesis.

The present invention relates to a hydrogel that is useful for promoting neurotization, osteogenesis and angiogenesis.

### TECHNOLOGICAL BACKGROUND

In the field of regenerative medicine and tissue engineering, the peripheral nervous system is still, even to this day, given little consideration in a context of bone tissue regeneration. However, biological, experimental and clinical data demonstrate interactions between the main events of bone reconstruction, namely its neovascularization, its innervation and bone neoformation.

Recent biological data obtained by the team of inventors (Silva et al, Cell Death and Disease, 2017 Dec. 13; 8(12):3209) demonstrated, by means of two-dimensional co-culture models of sensory neurons and mesenchymal cells, the impact on osteogenesis of the communication between these two cell types. However, there are currently no three-dimensional materials or matrices which make it possible to put these observations into practice, either experimentally or therapeutically, with a view to developing a new innovative material dedicated to bone regeneration.

It is in this context that the inventors have developed a new hydrogel, capable of recruiting neurons, in particular sensory neurons, and of housing other cell types, and more particularly bone-forming and endothelial cells.

### SUMMARY OF THE INVENTION

A first aspect of the invention relates to a hydrogel comprising:

i) an elastin-like polypeptide comprising at least one alkenylated residue; and

ii) a peptide capable of recruiting neuronal and/or endothelial cells, in particular an IKVAV peptide.

According to one particular embodiment, the hydrogel according to the invention comprises:

i) an elastin-like polypeptide comprising at least one alkenylated residue, in particular an alkenylated methionine;

ii) a peptide capable of recruiting neuronal and/or endothelial cells, in particular an IKVAV peptide; and

iii) a crosslinking polymer, in particular a crosslinking polymer with thiol end groups.

According to one particular embodiment, the elastin-like polypeptide is a polypeptide comprising at least one occurrence of the sequence VPGMG. In a nonlimiting manner, the elastin-like peptide can in particular be the MGTELAAASEFTHMW[VPGMG]₂₀ (ELP20), MW[VPGVGVPGMG(VPGVG)₂]₅ (ELPM20) or MW[VPGVGVPGMG(VPGVG)₂]₁₀ (ELPM40) polypeptide.

According to one particular embodiment, the peptide is an IKVAV peptide, in particular an IKVAV peptide of formula Cys-{Beta-Ala}-Ile-Lys-Val-Ala-Val-{Beta-Ala}-Cys.

In another variant, the crosslinking polymer, in particular a crosslinking polymer with thiol end groups, is a multi-arm polymer. More particularly, the crosslinking polymer may be a 4-arms poly(ethylene glycol), in particular a 4-arms poly(ethylene glycol) with thiol end groups, in particular a 4-arms poly(ethylene glycol) PEG having an average molecular weight comprised between 10 and 30 kDa, more particularly a 4-arms PEG comprising 4 arms with thiol end groups having an average molecular weight comprised between 10 and 30 kDa. The 4-arms poly(ethylene glycol), in particular the 4-arms poly(ethylene glycol) with thiol end groups, can in particular have an average molecular weight of 20 kDa. In the context of the present invention, the average molecular weight is a molecular weight in weight.

In one particular embodiment, the crosslinking polymer with thiol end groups, the elastin-like polypeptide comprising an alkenylated methionine residue, and the IKVAV peptide are present in the hydrogel in an equimolar thiol/alkene ratio.

According to another particular embodiment, the concentration of the hydrogel is between 5 and 15% by density (w/v), in particular between 7 and 8% (w/v).

In another embodiment, the storage modulus G′ of the hydrogel is between 1 and 5, preferably between 1 and 1.5 kPa.

The hydrogel according to the invention may also comprise at least one biologically active agent, in particular at least one growth factor.

Another aspect of the invention relates to the hydrogel described in the present application, for use as a medicament.

According to another aspect, the invention relates to the hydrogel described in the present application, for use in a bone regeneration method.

Moreover, the invention also relates to an in vitro cell culture method, comprising the culturing of cells in a hydrogel as defined in the present application.

### DESCRIPTION OF THE FIGURES

FIG. 1. A) Schematic representation of the components of the hydrogel and of the production method; B) photograph showing an ELPM40+PEG hydrogel. Bar=1 mm.

FIG. 2. rheological characterization of the ELPM40+PEG hydrogels at various final concentrations at 37° C. The elastic moduli (G′) for each concentration are indicated in the figure.

FIG. 3. scanning electron microscopy of (A) ELPM40+PEG, (B) ELPM40+25% IKVAV, (C) ELPM40+25% VKAIV, (D) ELPM40+50% IKVAV, (E) ELPM40+50% VKAIV showing the pore structure of these hydrogels. F) Quantification of the pore size: the 25% of adhesion peptide compositions have pores smaller than the other compositions.

FIG. 4. in vitro analysis of the degradation of the hydrogels by determination of free amines in solution after incubation with proteinase K (0.5 U/mL) for 7 h.

FIG. 5. metabolic activity of endothelial cells (ECs), bone marrow mesenchymal stromal cells (BMSCs) and sensory neurons (SNs). All hydrogel compositions allowed the attachment and culture of the three cell types.

FIG. 6. Morphology of the endothelial cells 7 days after culture thereof in (A) ELPM40+PEG, (B) ELPM40+25% IKVAV, (C) ELPM40+25% VKAIV, (D) ELPM40+50% IKVAV and (E) ELPM40+50% VKAIV. In all the compositions, the cells were able to enter and migrate within the hydrogels and to form various structures.

FIG. 7. BMSCs associated with (A) ELPM40+PEG, (B) ELPM40+25% IKVAV, (C) ELPM40+25% VKAIV, (D) ELPM40+50% IKVAV and (E) ELPM40+50% VKAIV. The cells exhibit a spheroid morphology with all the hydrogel compositions. (F) Detail of cells immersed in an ELPM40+50% IKVAV hydrogel showing two connected nuclei (indicated by the white arrow), suggesting that the cells can proliferate within the gel.

FIG. 8. Morphology and diffusion of sensory neurons cultured in (A) ELPM40+PEG, (B) ELPM40+25% IKVAV, (C) ELPM40+25% VKAIV, (D) ELPM40+50% IKVAV and (E) ELPM40+50% VKAIV. Bars 100 μm. (F) Average length of the neurites, measured for all the hydrogel compositions.

FIG. 9. Gene expression in BMSCs cultured in an osteogenic medium for 7 days, in combination with various hydrogels. The expression is represented relative to the ELPM40+PEG composition, used as a control.

FIG. 10. Tek gene expression after 7 days of culture of endothelial cells in ELPM40+PEG, ELPM40+25% IKVAV, ELPM40+25% VKAIV, ELPM40+50% IKVAV and ELPM40+50% VKAIV compositions.

FIG. 11. Subcutaneous evaluation of an ELPM40+50% IKVAV and ELPM40+50% VKAIV composition after 11 and 26 days. (A) histological sections were analyzed to determine i) the inflammatory potential (HE), ii) the capacity to induce angiogenesis (CD31 immunohistochemistry) and iii) the innervation (tubulin βIII immunohistochemistry (β3T)). Bars=50 μm for HE and CD31. Magnifications of the CD31 and β3T stainings are shown, bars=20 μm. (B) quantification of the vessels formed in the region surrounding the area of implantation of an ELPM40+50% and ELPM40+50% VKAIV IKVAV composition.

### DETAILED DESCRIPTION OF THE INVENTION

The present invention relates to a biocompatible hydrogel capable of promoting neurotization, osteogenesis and angiogenesis. This hydrogel is characterized in that it comprises an elastin-like polypeptide comprising at least one alkenylated methionine residue; and a peptide capable of recruiting neuronal and/or endothelial cells, in particular an IKVAV peptide. More particularly, the hydrogel according to the present invention is characterized in that it comprises i) an elastin-like polypeptide comprising at least one alkenylated methionine residue, and ii) a peptide capable of recruiting neuronal and/or endothelial cells, in particular an IKVAV peptide, and iii) a crosslinking polymer, in particular a crosslinking polymer with thiol end groups.

The hydrogel according to the invention can in particular be formed by means of a crosslinking polymer with thiol end groups. In the context of the present invention, the term “crosslinking polymer with thiol end groups” is intended to mean a polymer which has at least one free SH thiol function before formation of the hydrogel, that is to say before being brought into contact with the elastin-like peptide. According to one particular embodiment, when the thiol/alkene ratio is equimolar or when the thiol/alkene ratio is greater than 1 (more thiols than alkene), the polymer with thiol end groups in the hydrogel no longer has a free SH thiol function after reaction with the ELP. According to another embodiment, when the thiol/alkene ratio is less than 1 (thiol in deficit or alkene in excess), the polymer with thiol end groups, in the hydrogel, can have free SH thiol functions after reaction with the ELP. Said polymer is selected from polymers which allow the formation of a biocompatible hydrogel, in the sense that it is not toxic to cells. It also advantageously allows the diffusion of oxygen and of nutrients, and also that of carbon dioxide and of metabolic waste in order to feed the cells and to enable their survival. The polymers of the hydrogel solution may be of natural origin, such as extracellular matrix proteins, or synthetic origin, such as poly(ethylene glycol) (PEG), poly(oxazoline) (POx) or poly(sarcosine) (PSar). According to one embodiment, the crosslinking polymer is more particularly a multi-arm polymer, in particular a linear polymer or a multi-arm polymer having at least three arms, more particularly at least four arms, it being possible for said multi-arm linear polymer to comprise a thiol group at each of its ends. Thus, the crosslinking polymer may more particularly be a multi-arm polymer, in particular a linear polymer or a multi-arm polymer having at least three arms, more particularly at least four arms, said multi-arm linear polymer comprising a thiol group at each of its ends. According to one particular embodiment, the multi-arm polymer is a 4-arms polymer, that can in particular comprise a thiol group at the end of each of its arms. Mention may in particular be made of polymers of poly(ethylene glycol) (or PEG) type which are linear or which comprise three or four arms, or more than four arms, more particularly four arms. According to one embodiment, use is made of polymers of poly(ethylene glycol) (or PEG) type which are linear or which comprise three or four arms, or more than four arms, more particularly four arms, each end of the linear PEGs comprising a thiol group, or each arms of the multi-arm PEGs comprising a thiol group at their ends. In one embodiment, the hydrogel comprises a 4-arm PEG, each comprising a thiol group at its end, the average molecular weight of said PEG being between 1 and 100 kDa, more particularly between 10 and 30 kDa, the PEG having even more particularly an average molecular weight of 20 kDa.

The second component of the hydrogel according to the invention is an elastin-like polypeptide (ELP) comprising at least one alkenylated methionine residue. Polypeptides of this type, the method for producing them by genetic engineering, and their purification are known to those skilled in the art who can in particular refer to international application WO2017021334 and to the articles of Petitdemange et al. (Biomacromolecules. 2017 Feb. 13; 18(2):544-550) and Petitdemange et al. (Bioconjug Chem. 2017 May 17; 28(5):1403-1412). In the context of the present invention, the term “alkenylated methionine residue” means that the side chain of the methionine residue is covalently bonded to a group comprising an alkene group, that is to say comprising at least one double bond between two carbon atoms. Preferably, the term “alkene group” refers to the presence of a —CH═CH2 group in the group bonded to the methionine residue. According to one particular embodiment, the methionine group is bonded to the group of formula (I):

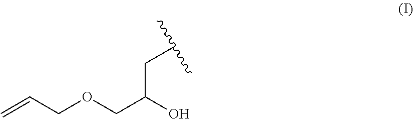 [SMILES: *CC(O)COCC=C]

According to one embodiment, the synthesis of an alkenylated ELP by means of the group of formula (I) can be carried out by chemoselective thioalkylation at the methionine side chains using an ally! glycidyl ether according to the procedure described in Petitdemange et al. (Bioconjug Chem. 2017 May 17; 28(5):1403-1412).

According to one embodiment, the alkenylated ELP used in the context of the present invention comprises at least one occurrence of the amino acid sequence VPGMG in which the methionine residue is alkenylated.

In one embodiment, the alkenylated ELP has a structure of formula (II)

Z-[VPGXG]ₙ—OH (II)

wherein:

Z is a peptide comprising between 1 and 20 amino acids;

X represents a glycine residue, a valine residue or an alkenylated methionine residue, in particular an alkenylated methionine residue of formula (III):

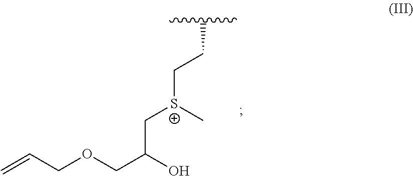

n is an integer between 1 and 200, more particularly between 10 and 200, even more particularly between 15 and 50, in particular between 20 and 40; and

wherein the ratio between the molar ratio of valine/alkenylated methionine in position X is between 0:1 and 10:1, more particularly between 1:1 and 5:1, said ratio being more particularly 3:1.

According to one embodiment, X represents a glycine residue or an alkenylated methionine residue, in particular an alkenylated methionine residue of formula (III).

According to another embodiment, which is preferred, X represents a valine residue or an alkenylated methionine residue, in particular an alkenylated methionine residue of formula (III).

According to one embodiment, n is an integer comprised between 30 and 50, in particular between 35 and 45, n being more particularly equal to 35, 36, 37, 38, 39, 40, 41, 42, 43, 44 or 45. More particularly, n is equal to 40.

According to one particular embodiment, Z is a peptide of which the amino acid residue at the amino-terminal end is a methionine.

According to one particular embodiment, Z does not comprise the amino acid sequence IKVAV. According to another embodiment, the amino acids included in Z immediately upstream of the [VPGXG]ₙ unit correspond to the MW dipeptide. Z can in particular consist of the MW dipeptide or can comprise this dipeptide. By way of illustration, the ELP20 peptide described below comprises a sequence Z wherein the MW dipeptide is at its C-terminal end in the sequence MGTELAAASEFTHMW.

According to one embodiment, the ELP employed is a peptide of the formula Z-[VPGXG]ₙ wherein X is an alkenylated methionine.

According to another embodiment, the ELP employed is a peptide of formula Z-[VPGXG]ₙ wherein the molar ratio of valine/alkenylated methionine in position X is between 0:1 and 10:1, more particularly between 1:1 and 5:1, said ratio being more particularly 3:1.

According to another embodiment, the ELP employed is a peptide of formula Z-[VPGVGVPGMG(VPGVG)₂]ₓ, in particular MW[VPGVGVPGMG(VPGVG)₂]ₓ wherein x is an integer comprised between 2 and 15, more particularly between 5 and 10.

According to one embodiment, the ELP employed is derived from a peptide chosen from the peptide MGTELAAASEFTHMW[VPGMG]₂₀ (ELP20), the peptide MW[VPGVGVPGMG(VPGVG)₂]₅ (ELPM20) or the peptide MW[VPGVGVPGMG(VPGVG)₂]₁₀ (ELPM40) described in WO2017021334, said peptide comprising at least one alkenylated methionine residue.

According to one particular embodiment, the ELP has the following structure:

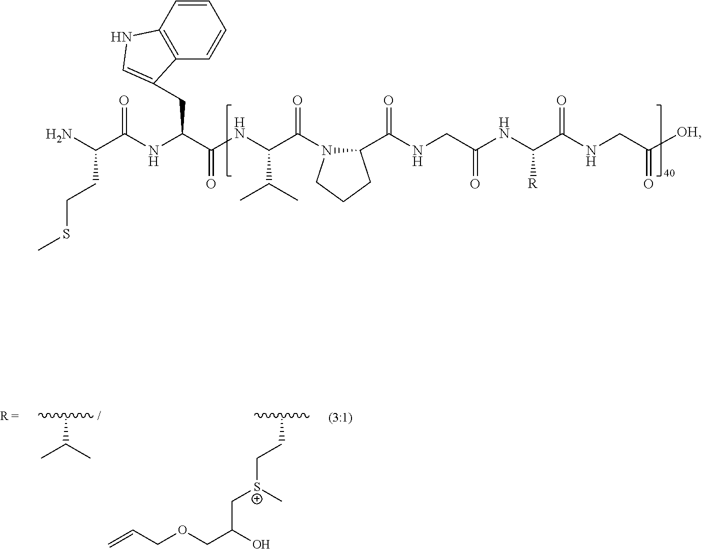

more particularly the structure MW[VPGVGVPGMₐG(VPGVG)₂]₁₀ (ELP-M(alkene)-40), wherein Mₐ represents the alkenylated methionine residue of formula (III) above.

According to one variant embodiment of all the ELPs described above, the amino-terminal methionine of said ELPs is an alkenylated methionine, in particular an alkenylated methionine of formula (III) above. By way of illustration, the ELP can in particular have the following structure:

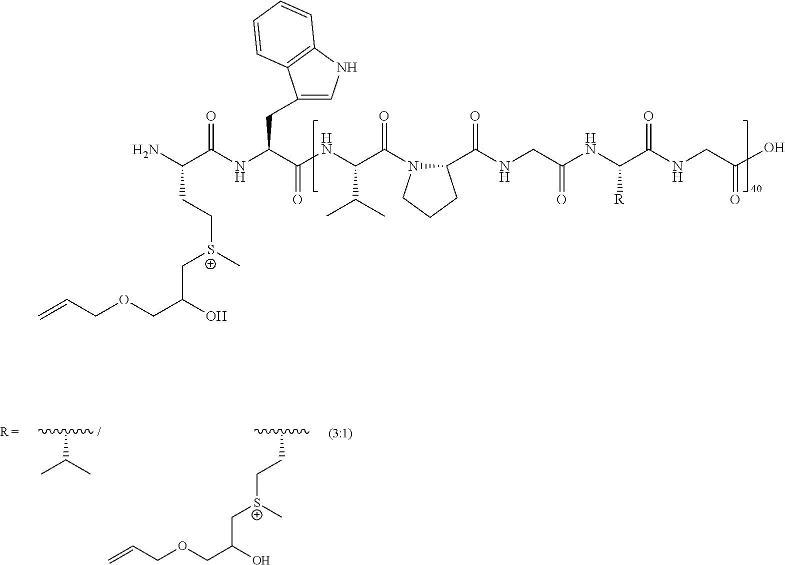

In one embodiment, this structure can be represented alternatively according to the following formula:

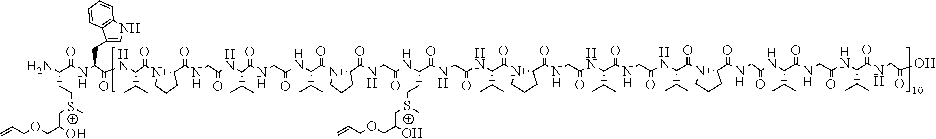

Component iii) of the hydrogel is a peptide capable of recruiting neuronal and/or endothelial cells. Mention may in particular be made, as peptides capable of recruiting endothelial cells, of the peptides REDV, RGD and GRGDSP derived from fibronectin, IKLLI, IKVAV, PDSGR and YIGSR, derived from laminin, and the peptide DGEA derived from collagen type I. As peptides capable of recruiting neuronal cells, mention may in particular be made of the peptides YIGSR, RNIAEIIKDI and IKVAV derived from laminin. According to one particular embodiment, the peptide capable of recruiting neuronal and/or endothelial cells is an IKVAV peptide, that is to say a peptide comprising the amino acid sequence IKVAV derived from laminin A. In one particular embodiment, the peptide capable of recruiting neuronal and/or endothelial cells included in the hydrogel of the invention is a peptide, in particular a peptide comprising the sequence IKVAV, comprising cysteine residues at each of its ends. The cysteine residues can be covalently bonded directly to the amino acid sequence IKVAV, or by means of spacers. According to one particular embodiment, the cysteine residues are bonded to the peptide capable of recruiting neuronal and/or endothelial cells, in particular to an IKVAV peptide, by means of a spacer, in particular a peptide spacer or pseudo peptide spacer. The spacer can in particular be an amino acid or an amino acid sequence (in particular a dipeptide or tripeptide), in particular a beta amino acid, more particularly a beta-Ala amino acid. Thus, according to one particular embodiment, the peptide capable of recruiting neuronal and/or endothelial cells is an IKVAV peptide of formula Cys-{spacer}-Ile-Lys-Val-Ala-Val-{spacer}-Cys, in particular the peptide of formula Cys-{Beta-Ala}-Ile-Lys-Val-Ala-Val-{Beta-Ala}-Cys.

The amount of the components of the hydrogel can vary to a large extent, provided that the resulting hydrogel promotes neurotization, osteogenesis and/or angiogenesis. According to one particular embodiment, the various components are in an amount which respects a thiol/alkene molar ratio of between 10:1 and 1:10, in particular between 5:1 and 1:5, in particular between 2:1 and 1:2. According to one particular embodiment, the thiol/alkene ratio is equimolar (i.e. it corresponds to a 1:1 molar ratio). It should be understood that the thiol groups are present on the crosslinking polymer and/or on the peptide capable of recruiting neuronal and/or endothelial cells, in particular an IKVAV peptide as described above. Thus, the hydrogel can comprise variable amounts of each of its components, while at the same time respecting the ratios defined above. For example, the thiol groups borne by the IKVAV peptide can represent between 10 and 75 mol % of all the thiol groups provided by the

IKVAV peptide and the crosslinking polymer, more particularly between 20 and 60 mol %, in particular between 25 and 50 mol % of all the thiol groups provided by the IKVAV peptide and the crosslinking polymer. According to one particular embodiment, the thiol groups borne by the IKVAV peptide represent 25 mol % of all the thiol groups provided by the IKVAV peptide and the crosslinking polymer. According to another particular embodiment, the thiol groups borne by the IKVAV peptide represent 50 mol % of all the thiol groups provided by the IKVAV peptide and the crosslinking polymer.

The proportions of the components of the hydrogel are also selected so as to obtain a hydrogel having rheological properties suitable for the development of cells, in particular neuronal, bone or endothelial cells, in particular neuronal cells. The invention thus makes it possible to very finely adjust the structure of the hydrogel to the cell type(s) of which the development must be promoted. According to one embodiment, the rigidity of the hydrogel corresponds to the storage modulus G′ parameter included in the following range: 1 kPa<G′<5 kPa, in particular 1 kPa<G′<1.5 kPa.

According to another particular embodiment, the concentration of hydrogel is between approximately 5 and approximately 15% by density (w/v), in particular between approximately 7 and approximately 8% (w/v), this density being more particularly equal to approximately 7.5% (w/v).

Moreover, the hydrogel according to the invention has a microporosity, and can comprise pores of average size ranging in particular between 5 and 20 μm, more particularly between 10 and 17 μm.

According to one particular embodiment, the hydrogel of the invention may comprise one or more other elements, other peptide sequences for targeting other functions, or also bioactive agents such as growth factors, in particular for stimulating even more the neurotization, osteogenesis and/or angiogenesis. However, according to one particular embodiment, the hydrogel is devoid of growth factors or contains them only in an amount of 0 to 10% by weight relative to the total weight of the hydrogel, more particularly 0 to 5%, more particularly still 0 to 1%, particularly 0 to 0.1% by weight relative to the weight of the hydrogel. As will be seen below, the hydrogel can also be used as a cell therapy carrier. Thus, a hydrogel as defined above can also comprise cells of therapeutic interest, for example stem cells, in particular stem cells induced towards a lineage of interest, hematopoietic stem cells, mesenchymal stromal stem cells derived from bone marrow or from adipose tissue, neuronal stem cells, or a mixture of cells of different lineages for stimulating a cell communication process. In one particular embodiment, the cells are stem cells, with the exclusion of human embryonic stem cells. The cells are introduced into the hydrogel after formation of the said hydrogel, by bringing the cells into contact with the hydrogel and culturing for a sufficient time (in particular for at least 1, 2, 3, 4, 5, 6 or at least 7 days) so that the cells can colonize the hydrogel.

The hydrogel according to the invention can also include nanoparticles of mineral components, in particular of hydroxyapatite or of calcium phosphate (in particular in an amount of between 10 and 40% (w/v)), in order to increase the osteogenic potential of the hydrogel.

The hydrogel of the invention can be produced by mixing these various components i) to iii), and any other optional element, such as growth factors. Components i) to iii) and the amount of these components i) to iii) are chosen in order to prepare a hydrogel having physical and support properties suitable for the problem addressed by its user. Advantageously, the formation of the hydrogel by crosslinking is carried out under the action of a stimulus such as a temperature or pH modification, or by means of a crosslinking agent, in particular a photosensitive crosslinking agent (or photoinitiator). By way of illustration, mention may thus be made of the induction of a photopolymerization by means of a photoinitiator such as the Irgacure 2959 compound, in particular used at a density of 0.5% (w/v) in the mixture, and activated by UV-visible light (λ=305-405 nm, in particular at 305 nm). In another variant, the photoinitiator can in particular be selected from lithium phenyl-2,4,6-trimethylbenzoylphosphinate (LAP) and riboflavin. LAP concentration can range from 0.005% to 0.5% (w/v) in the mixture, and the photoinitiation thereof can be triggered at a wavelength of between 365 and 475 nm.

According to one aspect, the invention relates to a hydrogel obtainable, or obtained, by means of a method comprising the following steps:

(a) mixing
i) an elastin-like polypeptide comprising at least one alkenylated methionine residue and
ii) a peptide capable of recruiting neuronal and/or endothelial cells, in particular an IKVAV peptide, and
optionally (iii) a crosslinking polymer with thiol end groups; and
iv) a polymerization initiator, in particular a photoinitiator (and optionally another component such as one or more growth factors); and
b) applying a stimulus, in particular light radiation, in particular UV radiation, in order to activate the polymerization.

Advantageously, as indicated above, the rheological properties of the hydrogel can be very finely defined. Moreover, the inventors have been able to show that the hydrogel according to the invention is degradable and has a pore structure. Finally, the inventors have been able to demonstrate that this hydrogel is suitable for culturing very different cell types, namely neurons, mesenchymal cells, bone cells, endothelial cells or progenitors thereof, that these cells can migrate within the structure of the hydrogel, and that it has no cytotoxic effect. It thus brings together all the advantageous properties useful to the development of a tool suitable for tissue regeneration.

The hydrogel according to the invention is thus capable of effectively supporting the in vitro culture of various cell types. Consequently, according to one particular embodiment, the invention relates to a new three-dimensional support capable of housing, in vitro, various cells of interest for bone regeneration, in particular neuronal, bone or endothelial cells. The invention thus provides those skilled in the art with a particularly advantageous 3D cell culture system which allows the cells to develop in a favorable environment, but also makes it possible to study the interactions of various cell types with one another. This parameter is important for studying the regeneration phenomena that can require complex dialogues between various cell types. The support of the invention can in particular be used to house bone-forming and endothelial cells, and for studying the angiogenic, osteogenic and neurotization effect in an in vitro cell culture method, comprising the culturing of cells in a support as defined above. The use of the support according to the invention can moreover comprise the addition of an agent to the culture, such as a growth factor or any other agent having a biological effect or potentially able to have a biological effect (candidate agent) for determining its effect on one or more parameters and cell responses, such as the growth of the cells, the induction of quiescence, of cell death, of protein secretion, or of other molecules, or of ions (in particular calcium ions, potassium ions), or also the expression of certain genes.

According to another aspect, the hydrogel of the invention is used in a treatment method, in particular as an implant. The hydrogel is in particular used in a regenerative medicine treatment method. It can in particular be used for stimulating the innervation of a tissue, in particular a bone tissue, and can in particular be used in bone engineering. Advantageously, the hydrogel according to the invention promotes neurotization, in particular in a regeneration context. More particularly, the hydrogel according to the invention can advantageously be used for recruiting and stimulating the sensory nervous system, more particularly for promoting bone regeneration. The hydrogel according to the invention can also be employed in order to optimize or restore the vascularization and innervation of a tissue.

According to another embodiment, the hydrogel can be used as a cell therapy carrier. Thus, a hydrogel as defined above, previously colonized with cells of therapeutic interest, for example by means of stem cells, in particular stem cells induced into a lineage of interest, hematopoietic stem cells, mesenchymal stromal stem cells derived from bone marrow or from adipose tissue, neuronal stem cells, or a mixture of cells of different lineages. Such a hydrogel can be used in a method of treatment by cell or tissue regeneration.

The hydrogel according to the invention may also be used for modifying implant systems, for improving their biocompatibility and their integration.

### EXAMPLES

### Production of the ELP-M(alkene)-40 Peptide

The article by Petitdemange et al. (Biomacromolecules. 2017 Feb. 13; 18(2):544-550. doi:10.1021/acs.biomac.6b01696. Epub 2017 Jan 27. PubMed PMID: 28075561) describes the construction of the expression vector for the MX[VPGVGVPGMG(VPGVG)₂]₁₀ peptide (ELPM40), its expression in E. coli, its isolation from bacterial lysates, its purification and its characterization.

The method for producing the ELP-M(alkene)-40 peptide, having the structure:

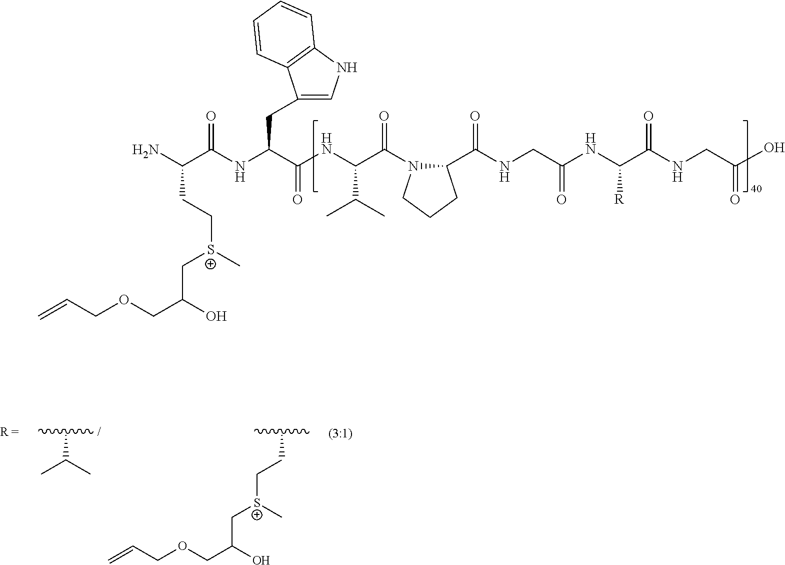

by chemoselective thioalkylation of the methionine side chains of the ELPM40 peptide using ally! glycidyl ether is described in Petitdemange et al. (Bioconjug Chem. 2017 May 17; 28(5):1403-1412. doi: 10.1021/acs.bioconjchem.7b00082. Epub 2017 Apr. 18. PubMed PMID: 28381088).

In the remainder of the experimental section, the term ELPM40 is used to denote the ELP-M(alkene)-40 peptide.

### Development of a Composite Hydrogel

### Production of the Hydrogel

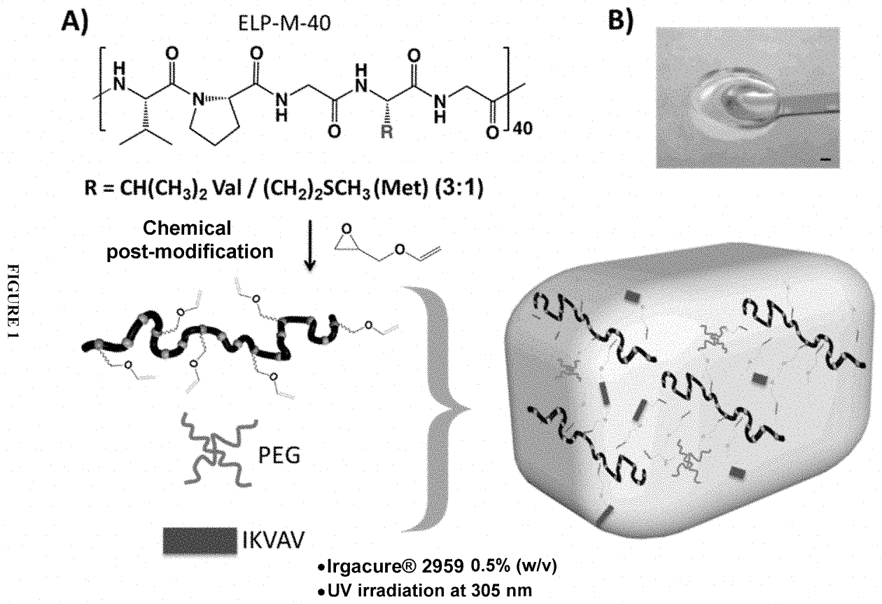

The hydrogels produced contain the ELPM40 peptide, a PEG with thiol end groups (SH-PEG, 20 kDa; JenKem, USA) and the Cys-{Beta-Ala}-Ile-Lys-Val-Ala-Val-{Beta-Ala}-Cys (IKVAV) adhesion peptide or a randomized version Cys-{Beta-Ala}-Val-Lys-Ala-Ile-Val-{Beta-Ala}-Cys (VKAIV), at an equimolar thio/alkene ratio. Photopolymerization was carried out using the Irgacure 2959 photoinitiator (0.5% w/v) exposed to UV light having a wavelength of 305 nm for 8 minutes, subsequent to a thiol-ene reaction. FIG. 1 shows a schematic representation of the hydrogel thus obtained.

Various weight ratios of ELP and PEG were tested in order to obtain optimal rigidity and sensory neuron attachment and neurite outgrowth. Briefly, the SH-PEG was replaced with the adhesion peptide in various proportions. The compositions tested are the following:

(i) ELPM40+PEG, wherein 100% (mol) of the thiol groups are represented by the SH-PEG,

(ii) ELPM40+25% IKVAV or VKAIV, wherein 25% (mol) of the thiol groups are represented by the adhesion peptide, and

(iii) ELPM40+50% IKVAV or VKAIV, wherein 50% (mol) of the thiol groups are represented by the adhesion peptide.

### Rheological Properties

The rheological properties of the hydrogels were evaluated after 24 h of soaking in PBS, by measuring the frequency dependence of the elastic (G′) and loss (G″) moduli. Frequency sweeps were carried out on ELPM40+PEG compositions at various hydrogel concentrations, i.e. at 5%, 7.5%, 10% and 15% (w/v). Measurements were carried out at 37° C.

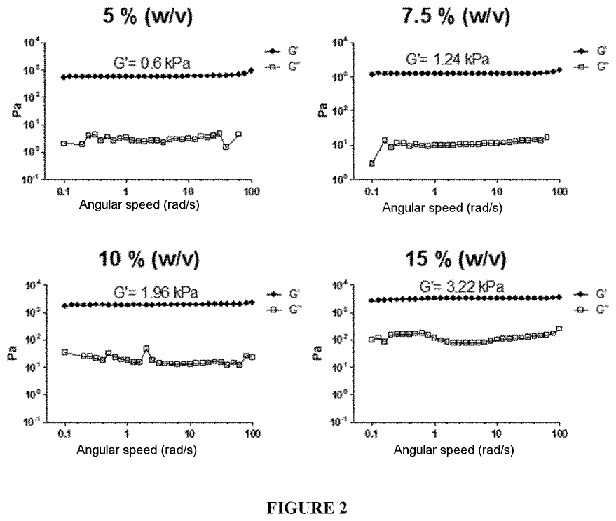

It was possible to show that the elastic modulus increased proportionally to the final concentration of the hydrogel (FIG. 2).

### Structural Analysis by Scanning Electron Microscopy

An analysis by scanning electron cryomicroscopy was carried out to determine the pore size of the hydrogels produced at various concentrations, and to visualize their structure.

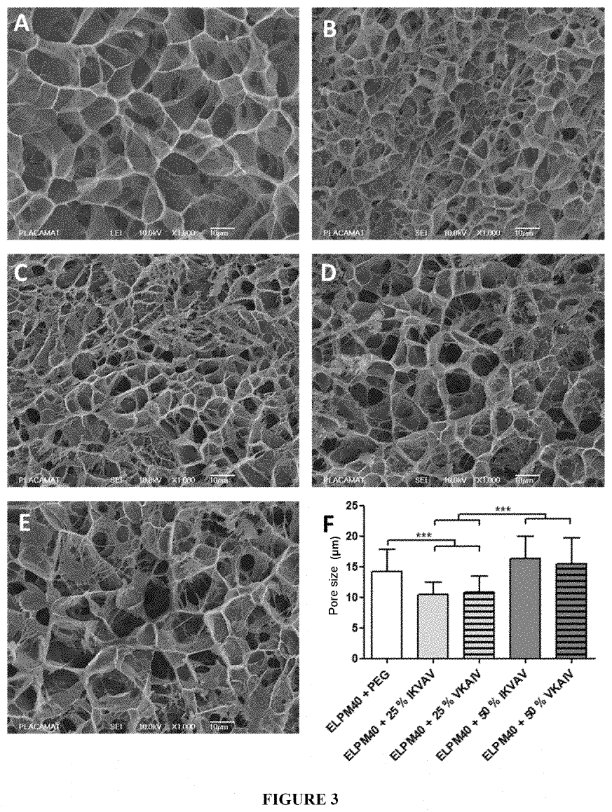

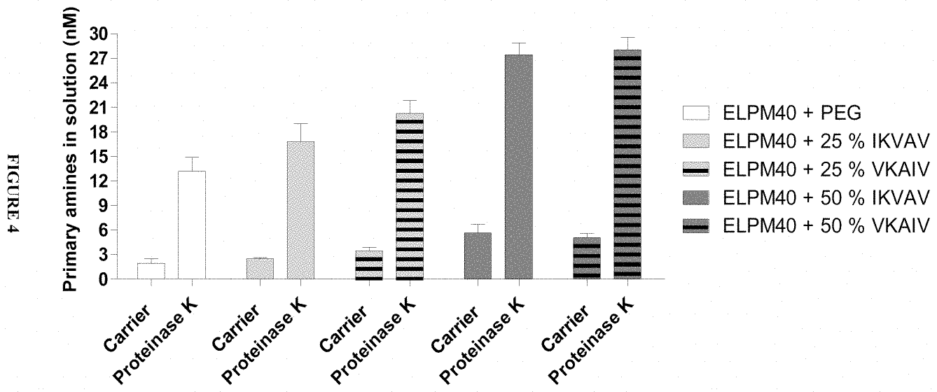

All the hydrogel compositions have a pore structure (FIGS. 3A-E), with a pore size ranging from 10.49±1.61 μm (ELPM40+25% IKVAV) to 16.39±2.81 μm (ELPM40+50% IKVAV). The hydrogel compositions comprising 25% of adhesion peptide have smaller pore sizes in comparison with the compositions obtained under the other conditions (FIG. 3F).

### In Vitro Degradation Analysis of the Hydrogel

15 μL of hydrogel were incubated in 150 μL of a proteinase K solution (0.5 U/mL) (Amresco, #0706) in a 50 mM Tris Base buffer containing 1 mM EDTA, 5 mM CaCl₂ and 0.5% (v/v) Triton X-100 (pH 8.0) for 7 h at 37° C. The content of free amine groups, expressed by the number of free amine groups present per 1000 amino acids (n/1000) was determined using 2,4,6-trinitrobenzenesulfonic (TNBS) acid according to the procedure described in Gilbert et al. J Biomed Mater Res 1990, 24, 1221. The content of free amine groups was calculated using a molar absorption coefficient of 14600 L/mol/cm for trinitrophenyl lysine (Wang et al., Biochim Biophys Acta 1978, 544, 555).

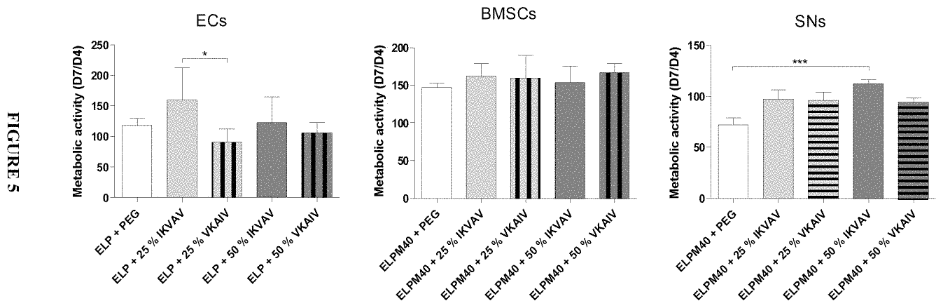

After incubation with proteinase K, free primary amines in solution could be detected for all hydrogel compositions (FIG. 4), indicating that the crosslinking process did not interfere with the in vitro degradation properties. This is an important property for materials having biomedical applications. It must be possible for various cell types to enter into the structure of the hydrogel, and to migrate therein. The most common cell migration mechanism is the secretion of proteases which digest the hydrogel structure at a slow rate, thereby enabling the colonization of the hydrogel by the cells.

### Biological Evaluation with Primary Cells

### Isolation of Cells and Culture

Primary sensory neurons (SNs) were obtained from dorsal root ganglions (DRG) from 6- to 10-week-old Wistar rats, according to a procedure described by Malin et al., Nat Protoc 2007, 2, 152.

Bone marrow-derived mesenchymal stromal cells (BMSCs) were isolated from 6- to 10-week-old Wistar rats. Briefly, the femurs and tibias of the animals were removed and cut at their ends to expose the bone marrow. The bones were transferred into 1.5 mL tubes and then centrifuged at 1500×g for 30 s so as to expel the bone marrow therefrom. The pellets obtained were then resuspended in DMEM with a low glucose content (Gibco) supplemented with 10% (v/v) of fetal calf serum (PANTM-Biotech, Aidenbach, Germany) and with 1% (v/v) of penicillin/streptomycin, then passed through 16G and 21G needles 4 to 6 times. The content obtained from a femur and from a tibia was then seeded in a 75 cm² flask and incubated in a humidified incubator (37° C. and 5% CO₂). The culture medium was changed twice a week in order to remove non-adherent cells. Adherent cells were cultured up to a confluency of 90%, then transferred into containers with a larger surface area. The cells were used up to passage P3. When they were combined with the hydrogels, the BMSCs were cultured in an osteogenic medium, which corresponds to the culture medium mentioned above, supplemented with 10⁻⁹ M of dexamethasone (Sigma-Aldrich), 10 mM of β-glycerophosphate (Sigma-Aldrich) and 50 μg/mL of ascorbic acid (Sigma-Aldrich).

The bone marrow-derived endothelial stem cells (ECs) were acquired from Cell Biologics (catalogue number RA-6221). The cells were cultured in an EGM-2 MV medium (Lonza-Verviers, France) on plates coated with gelatin (2%) containing all the supplements of the kit and 5% (v/v) of fetal calf serum (Gibco Life Technologies, Karlsruhe, Germany) and incubated at 37° C. in a humid atmosphere, with 5% of CO₂. The cells were transferred onto another plate coated with gelatin (2%) when they reached 90% confluency.

The three cell types were studied on the hydrogels of the invention because of their advantage in tissue engineering: (i) the BMSCs because of their osteoblast differentiation potential, (ii) the ECs for their major role in angiogenesis, and (iii) the sensory neurons in order to demonstrate the capacity of the nervous tissue to adhere and to allow the growth of neurites in the hydrogels.

Each cell type was characterized before its culture in the hydrogels of the invention.

### Cellular Metabolic Activity of Each Cell Type in the Hydrogels

The metabolic activity of the cells was determined using a test based on the use of resazurin (O'Brien et al., Eur J Biochem 2000, 267, 5421). Briefly, the cells were seeded in hydrogels at 7.5% (w/v) at a density of 20000 cells/cm² in a 96-well plate. 150 μL of cell medium containing resazurin (0.01 mg/mL) were added to each well and the microplate was incubated at 37° C. for 3 h. 100 μL of supernatant were then transferred into another 96-well microplate, and the fluorescence was measured (exc=530 nm, em=590 nm, Victor X3, Perkin Elmer). The metabolic activity was measured on days 4 and 7 (n=5).

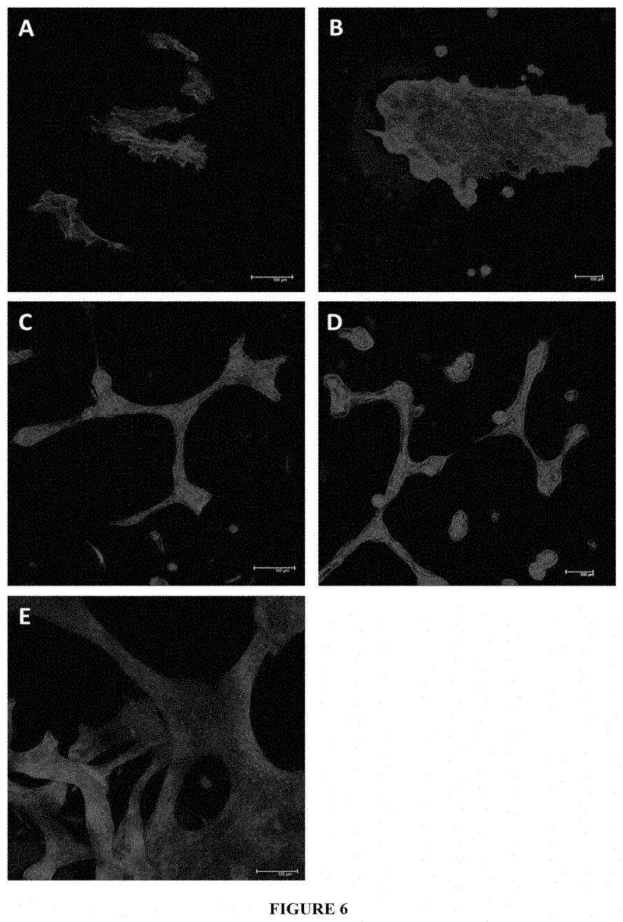

FIG. 5 presents the results obtained. The hydrogels allow the attachment and culture of the various primary cells tested without a cytotoxic effect. For ECs, the metabolic activity was greater in the ELPM40+25% IKVAV hydrogel, compared with the corresponding random peptide control. For SNs, the ELPM40+50% IKVAV hydrogel induces a greater metabolic activity compared to the ELPM40+PEG composition.

Other experiments also showed that hydrogels comprising another ELP sequence linked to PEG do not induce an inflammatory response when they are implanted subcutaneously in mice.

### Confocal Microscopy of Cell-Containing Hydrogels

BMSC, EC and SN cells were seeded in hydrogels at a concentration of 20000 cells/cm² and cultured for 7 days.

After culture, SNs were fixed at 4° C. for 30 minutes with 4% (w/v) of formaldehyde, permeabilized with triton X-100 at 0.1% (v/v) for 30 minutes at 4° C., and blocked with 1% (w/v) BSA for 1 h. An anti-beta III tubulin primary antibody was used for the detection of the cells. For BMSCs and ECs, actin filaments were labelled with phalloidin conjugated with Alexa Fluor 568. The morphology of the SNs was visualized using a confocal microscope (SPE, Leica Microsystems). Neurite length was quantified using the ImageJ software, with the “simple neurite tracer” tool. Neurites were measured by tracing a path from the soma to the visible end, then the length was converted into μm.

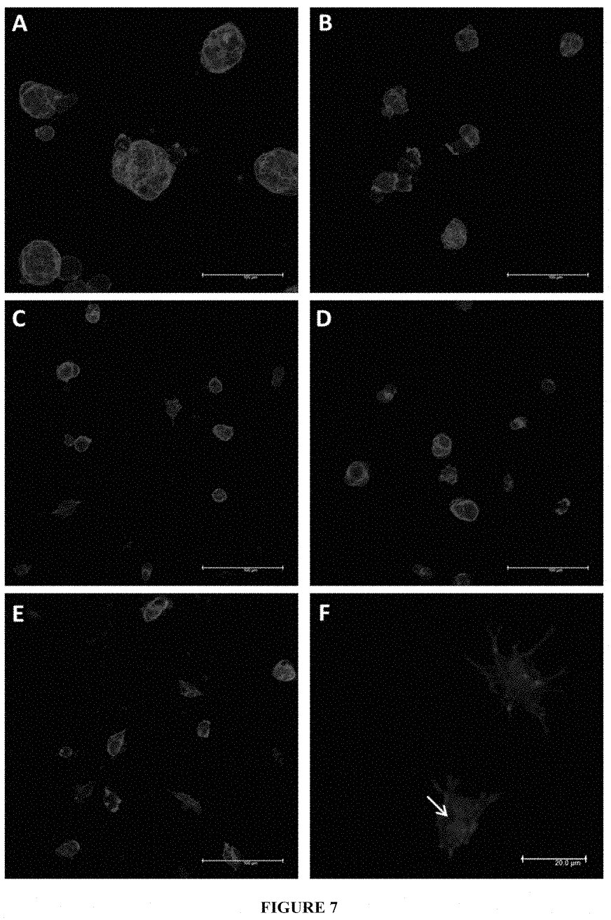

After 7 days of culture, ECs penetrated the hydrogels and formed therein stable branched structures (FIG. 6). These structures are important for the delivery of oxygen and nutrients to the vascularized tissue. It was possible to observe that the branched structures formed in the ELPM40+50% IKVAV hydrogel and in the two compositions comprising the random peptide. Although the random sequence of the adhesion peptide does not have the same adhesion function as the IKVAV peptide, in the hydrogel compositions containing the random peptide, no morphological difference was observed compared with the ELPM40+50% IKVAV composition. It is possible that this phenomenon is due to the increase in positive charges due to the lysin residues.

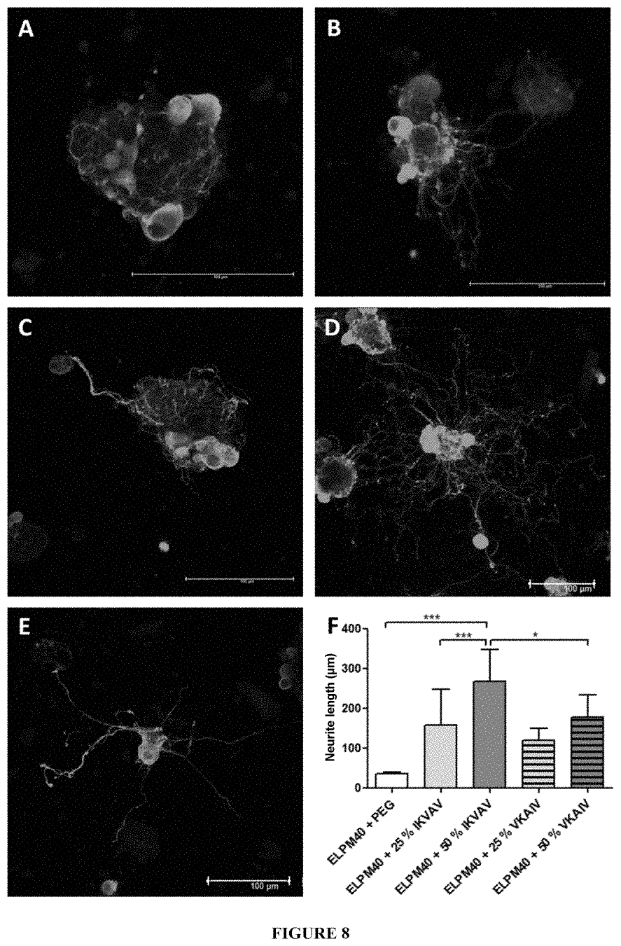

When BMSCs were combined with the hydrogels, they developed into spheroid aggregates for all the compositions (FIG. 7). Our study shows that, for the ELPM40+50% IKVAV composition, some BMSCs entered the structure of the hydrogel, and formed branches. A few cells were binucleated, with a discrete connection between the two nuclei, suggesting that the cells can divide inside the gel (FIG. 7F).

As regards SNs, the cells exhibited a distinct behavior depending on the hydrogel composition (FIG. 8). In ELPM40+PEG, the cells formed larger aggregates, and the neurites were not dispersed in the structure of the hydrogel, but instead surrounded the cell body (FIG. 8A). When the IKVAV sequence is added to the hydrogel composition, the neurites can spread. In the ELPM40+50% IKVAV composition, the cells adopted a more disperse distribution with a vast and branched complex network of neurites compared with the other compositions (FIG. 8D). The measurement of the neurite length confirms this morphological observation. Indeed, the SNs cultured in the ELPM40+50% IKVAV composition were able to form longer neurites (FIG. 8F).

It is important to note that the culture media used in this study do not contain growth factors (in particular NGF), this being for the purpose of analyzing the specific impact of the hydrogel on the structure and the expansion of the neurites. According to our results, the average neurite length in the ELPM40+50% IKVAV composition is 266.44±63.95 μm, which is equivalent to what had been measured in other studies carried out in 2D where the culture medium was supplemented with NGF.

### Molecular Analysis of Osteo-Specific Markers Expressed in BMSCs in the Hydrogel

Total RNAs were extracted from the BMSCs using the RNeasy® Plus Micro Kit (Qiagen, Hilden, Germany) according to the manufacturer's protocol. 100 ng of total RNAs obtained from 4 wells were reverse transcribed to cDNA using the Maxima Reverse Transcriptase kit (Thermo Scientific™, Thermo Fisher Scientific, Waltham, Mass., USA), according to the manufacturer's protocol. RT-PCR reactions were carried out, using the CFX Connect™ Real-Time PCR Detection System (Bio-Rad Laboratories, Hercules, Calif., USA) and analyzed using the CFX Manager™ software, version 3.0 (Bio-Rad Laboratories). The primers used are the following (SEQ ID NOs: 1 to 12):

Runx2
F:
5′ CCTTCCCTCCGAGACCCTAA 3′
and
R:
5′ ATGGCTGCTCCCTTCTGAAC 3′,
Sp7
F:
5′ TGCTTGAGGAAGAAGCTCACTA 3′
and
R:
5′ GGGGCTGAAAGGTCAGTGTA 3′,
Ctnnbl (βcat)
F:
5′ GAAAATGCTTGGGTCGCCAG 3′
and
R:
5′ CGCACTGCCATTTTAGCTCC 3′,
Bspl (Opn)
F:
5′ GAGTTTGGCAGCTCAGAGGA 3′
and
R:
TCTGCTTCTGAGATGGGTCA 3′,
Smadl
F:
5′ ATGGACACGAACATGACGAA 3′
and
R:
5′ GCACCAGTGTTTTGGTTCCT 3′,
Rplp0
F:
5′ CACTGGCTGAAAAGGTCAAGG 3′
and
5′ GTGTGAGGGGCTTAGTCGAA 3′,
and
Tek
F:
5′ CCACAGATAGAGGATTTGCCAG 3′
and
R:
5′ AAGTCATTTGGTTGGAGCACTG 3′.

The expression was quantified using the threshold cycle (Ct) values and mRNA expression levels were calculated according to the 2^{-DDCt} method.

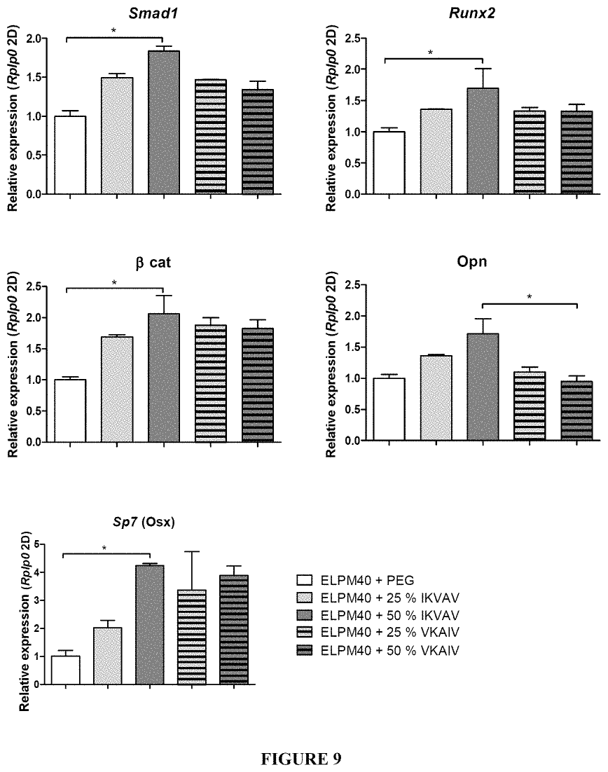

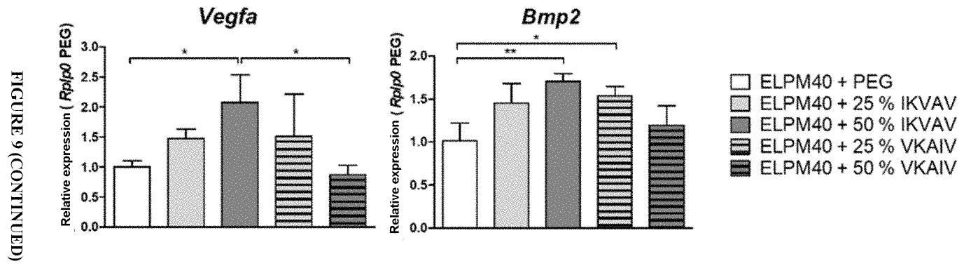

To determine whether the hydrogel compositions can support the osteogenic differentiation of BMSCs, a group of osteogenic markers of early differentiation (Runx2 and Sp7) and of late differentiation (Opn), were analyzed, as were genes linked to the triggering of the osteogenic signaling pathways (Smadl and βcat). Moreover, key factors for the bone repair process, which induce vascularization and osteoblast differentiation: vegfa and bmp2, respectively, were analyzed (FIG. 9).

After 7 days of culture in an osteogenic medium, an increase in Runx2 and Sp7 expression is observed in the ELPM40+50% IKVAV composition compared with ELPM40+PEG, and Opn expression is increased in the ELPM40+50% IKVAV composition compared with the random peptide control (FIG. 9). When the signaling pathways that could trigger osteogenic differentiation were analyzed, the expression of Smadl and βcat was increased in ELPM40+50% IKVAV compared with ELPM40+PEG, suggesting that the two signaling pathways play a role in BMSC differentiation associated with these hydrogels. These results therefore show that the ELP peptides can represent very good substrates promoting BMSC osteogenesis. Moreover, expression levels of all the genes tested in this study increased in a dose-dependent manner relative to the concentration of the IKVAV peptide. The overexpression of the genes which trigger and act directly at various stages of osteogenic differentiation supports the role of the ELPM40+50% IKVAV composition in the differentiation of BMSCs towards the osteogenic lineage. The expression of the Vegfa angiogenic factor increasd with the ELPM40+50% IKVAV composition compared with ELPM40+PEG or ELPM40+50% VKAIV. Bmp2 expression was increased in the presence of ELPM40+25%. Interestingly, the expression of all the genes studied was increased with the ELPM40+50% IKVAV composition compared with the ELPM40+PEG composition, and this increase was proportional to the IKVAV concentration.

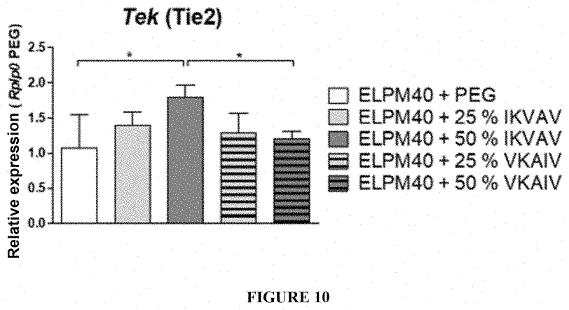

In order to determine the pro-angiogenic potential of the hydrogels of the invention, rat bone marrow primary endothelial cells were cultured in various hydrogel compositions. After 7 days of culture, the expression of Tek, which plays a role during vessel formation, was evaluated (FIG. 10). An overexpression of Tek was observed with the composition comprising 50% of IKVAV in comparison to those containing PEG or the random VKAIV peptide (p<0.05).

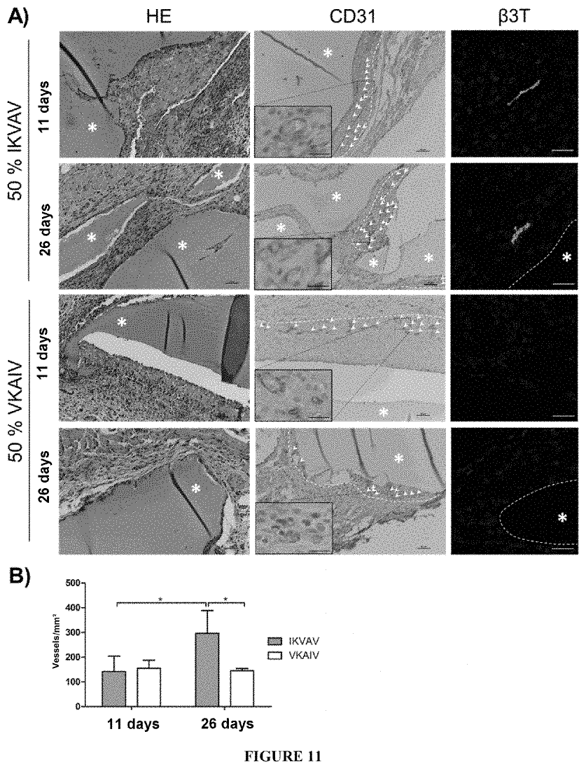

Finally, the ELPM40+50% IKVAV or VKAIV composition was implanted subcutaneously (FIG. 11). Neither triggered any major inflammation signals, as shown by the absence of multinuclear giant cells. When the blood vessels were quantified in the region surrounding the implantation area, a higher vessel density was observed with the composition containing IKVAV compared with that containing the VKAIV peptide after 26 days of implantation, vessel density increasing with time. These results are supported by the data obtained in vitro and the endothelial cell gene expression, showing an increase in the expression of Tek in the compositions containing 50% of IKVAV.

In conclusion, functional hydrogels based on ELP-M(alkene)-40, SH-PEG and a synthetic peptide comprising the IKVAV adhesion sequence were produced. These hydrogels have the advantage of having finely adaptable rheological properties. For the needs of the present study, the selected hydrogels have a weight concentration suitable for SN growth. These hydrogels are moreover degradable in vitro, and have a pore structure.

Biological evaluation has shown that the hydrogels of the invention support the culture of EC, BMSC and SN cells, that those cells were able to migrate inside the hydrogel structure after 7 days of culture and that no cytotoxic effect of the hydrogels was observed. As regards the vascularization potential, ECs were able to form stable branched structures in compositions comprising 50% of adhesion peptide. As regards the osteogenic potential, the morphology of BMSCs was of spheroid organization type in all the compositions and, when the cells were cultured in an ELPM40+50% IKVAV composition, a set of genes important for osteogenic differentiation was overexpressed. Finally, as regards the neurotization potential, SNs cultured in the ELPM40+50% IKVAV composition exhibited a more complex neurite network and a greater neurite length, the latter being comparable to that obtained in other neurite cultures performed in media supplemented with NGF, which is a growth factor known to promote neurite expansion.

The data presented in this application show that the strategy proposed, which is based on specific hydrogels which are the first supports developed that allow vascularization, osteogenesis and neurotization without the presence of other cellular factors or of growth factors, exhibits advantageous characteristics for biomedical applications.

1-15. (canceled)

16. A hydrogel comprising:
i) an elastin-like polypeptide comprising at least one alkenylated residue; and
ii) a peptide capable of recruiting neuronal and/or endothelial cells.

17. The hydrogel according to claim 16, also comprising:
iii) a crosslinking polymer with thiol end groups before formation of the hydrogel.

18. The hydrogel according to claim 16, wherein the peptide ii) is an IKVAV peptide or is a peptide of formula Cys-{Beta-Ala}-Ile-Lys-Val-Ala-Val-{Beta-Ala}-Cys.

19. The hydrogel according to claim 16, wherein the elastin-like polypeptide is a polypeptide comprising at least one occurrence of the VPGMG sequence.

20. The hydrogel according to claim 19, wherein the elastin-like polypeptide is the MGTELAAASEFTHMW[VPGMG]₂₀ (ELP20) polypeptide, the MW[VPGVGVPGMG(VPGVG)₂]₅ (ELPM20) polypeptide or the MW[VPGVGVPGMG(VPGVG)₂]₁₀ (ELPM40) polypeptide.

21. The hydrogel according to claim 17, wherein the crosslinking polymer with thiol end groups is a multi-arm polymer.

22. The hydrogel according to claim 17, wherein the crosslinking polymer is a 4-arm poly(ethylene glycol) with thiol end groups, having an average molecular weight between 10 and 30 kDa in weight.

23. The hydrogel according to claim 17, wherein the crosslinking polymer with thiol end groups, the elastin-like polypeptide comprising an alkenylated methionine residue, and the IKVAV peptide are present in an equimolar thiol/alkene ratio.

24. The hydrogel according to claim 16, wherein the concentration of the hydrogel is between 5 and 15% by density (w/v).

25. The hydrogel according to claim 16, wherein the storage modulus G′ of the hydrogel is between 1 and 1.5 kPa.

26. The hydrogel according to claim 16, said hydrogel also comprising at least one biologically active agent.

27. The hydrogel according to claim 26, wherein said at least one biologically active agent is a growth factor.

28. A three-dimensional support capable of housing cells of interest comprising a hydrogel according to claim 16.

29. A method of bone regeneration in a subject comprising introducing a hydrogel according to claim 16 into bone in need of regeneration.

30. An in vitro cell culture method, comprising culturing cells in a hydrogel according to claim 16 or in a three-dimensional support comprising said hydrogel.


SEQUENCE LISTING (29 sequences)

SEQ ID NO: 1 - DNA, 20 bp, artificial
  primer
ccttccctccgagaccctaa

SEQ ID NO: 2 - DNA, 20 bp, artificial
  primer
atggctgctcccttctgaac

SEQ ID NO: 3 - DNA, 22 bp, artificial
  primer
tgcttgaggaagaagctcacta

SEQ ID NO: 4 - DNA, 20 bp, artificial
  primer
ggggctgaaaggtcagtgta

SEQ ID NO: 5 - DNA, 20 bp, artificial
  primer
gaaaatgcttgggtcgccag

SEQ ID NO: 6 - DNA, 20 bp, artificial
  primer
cgcactgccattttagctcc

SEQ ID NO: 7 - DNA, 20 bp, artificial
  primer
gagtttggcagctcagagga

SEQ ID NO: 8 - DNA, 20 bp, artificial
  primer
tctgcttctgagatgggtca

SEQ ID NO: 9 - DNA, 20 bp, artificial
  primer
atggacacgaacatgacgaa

SEQ ID NO: 10 - DNA, 20 bp, artificial
  primer
gcaccagtgttttggttcct

SEQ ID NO: 11 - DNA, 21 bp, artificial
  primer
cactggctgaaaaggtcaagg

SEQ ID NO: 12 - DNA, 20 bp, artificial
  primer
gtgtgaggggcttagtcgaa

SEQ ID NO: 13 - protein, 5 aa, artificial
  motif peptide
VPGMG

SEQ ID NO: 14 - protein, 20 aa, artificial
  ELP20
  REPEAT (16)..(20): 20 repetitions
MGTELAAASEFTHMWVPGMG

SEQ ID NO: 15 - protein, 17 aa, artificial
  ELPM20
  REPEAT (3)..(17): 5 repetitions
  REPEAT (13)..(17): 2 repetitions
MWVPGVGVPGMGVPGVG

SEQ ID NO: 16 - protein, 17 aa, artificial
  ELPM40
  REPEAT (3)..(17): 10 repetitions
  REPEAT (13)..(17): 2 repetitions
MWVPGVGVPGMGVPGVG

SEQ ID NO: 17 - protein, 9 aa, artificial
  peptide IKVAV
  misc_feature (2)..(2): bAla
  misc_feature (8)..(8): bAla
CAIKVAVAV

SEQ ID NO: 18 - protein, 5 aa, artificial
  peptide IKVAV
IKVAV

SEQ ID NO: 19 - protein, 5 aa, artificial
  peptide VKAIV
VKAIV

SEQ ID NO: 20 - protein, 5 aa, artificial
  peptide
  misc_feature (4)..(4): X = glycine or methionine alkenyl
VPGXG

SEQ ID NO: 21 - protein, 15 aa, artificial
  peptide
MGTELAAASEFTHMW

SEQ ID NO: 22 - protein, 4 aa, artificial
  peptide
REDV

SEQ ID NO: 23 - protein, 6 aa, artificial
  peptide
GRGDSP

SEQ ID NO: 24 - protein, 5 aa, artificial
  peptide
IKLLI

SEQ ID NO: 25 - protein, 5 aa, artificial
  peptide
PDSGR

SEQ ID NO: 26 - protein, 5 aa, artificial
  peptide
YIGSR

SEQ ID NO: 27 - protein, 10 aa, artificial
  peptide
RNIAEIIKDI

SEQ ID NO: 28 - DNA, 22 bp, artificial
  primer
ccacagatagaggatttgccag

SEQ ID NO: 29 - DNA, 22 bp, artificial
  primer
aagtcatttggttggagcactg